(19) United States
(12) Patent Application Publication
(10) Pub. No.: US 2009/0077751 A1
(43) Pub. Date: Mar. 26, 2009

(54) Therapeutic Movement Table

(76) Inventors: Allen H. Jacobson, Colorado Springs, CO (US); Eric M. Dinkins, Castle Rock, CO (US)

Correspondence Address: HANES & SCHUTZ, LLC, 102 SOUTH TEJON ST., SUITE 800, COLORADO SPRINGS, CO, 80903, US

(21) Appl. No.: 11/862,093

(22) Filed: Sep. 26, 2007

(51) Int. Cl. A47C 20/00; A61F 5/00

(52) U.S. Cl. 5/640; 5/632; 602/32

(57) ABSTRACT

Therapeutic apparatus for manipulating the limbs of the human body, comprising a platform having a longitudinal aspect for supporting, in a fixed position, a human body, an arch shaped track pivotally supported by and superimposed over the platform where the plane of the track intersects the platform longitudinally, at least one anchor manually or mechanically movable within the track and which anchor is attached to one end of a tensioning device whose other end is intended for connection to the limb of a patient. The tensioning device pulls tension on the limb while the anchor travels over a portion of the length of the track to manipulate the limb in extension, abduction or a combination of both movements, among others.

### FIELD OF THE INVENTION

The present invention relates to a table and associated mechanism for therapeutically manipulating the limbs of the human body.

### BACKGROUND OF THE INVENTION

Tightness and spasms of hamstring tendons and quadriceps muscles, as well as nervous and other muscular disorders of the upper and lower extremities, may cause discomfort, pain and restriction of motion. Certain manipulations of the limbs have been known to provide at least temporary relief from these symptoms. It has been found that stretching or applying tension to a limb prior to its therapeutic manipulation provides a distraction that inhibits the nerve stretch response of muscles, thus allowing for a greater achievable range of motion than is available without the tension force.

While these facts may be known, their appropriate application by a therapist is difficult, sometimes requiring significant strength and endurance. It is therefore the primary object of the present invention to provide a mechanical means for simultaneously applying a distraction force and therapeutically manipulating a human limb.

### SUMMARY OF THE INVENTION

The stretching table of the present invention comprises a platform on which the patient is positioned. Pivotally attached to the platform at each of its longitudinal ends are the extremities of a track that arches over the platform and movably carries an anchor attached to one end of a body tensioning member. The track lies in a plane that may be angularly tilted in both directions away from a position where the plane of the track is perpendicular to the platform. In the preferred form of the device of the present invention the platform comprises a frame that rotatably supports a bed portion on which the patient may sit or recline. The bed portion may be rotated so that its longitudinal axis is angularly disposed with respect to the longitudinal axis of the frame and to the plane of the track.

In operation, the free end of the tension member is attached to a limb of the patient being treated and tension or stretching is thereby applied to the limb, the body of the patient being restrained in a fixed position on the platform bed. Once tension is applied to the limb the therapist may raise the distal end of the limb by operating a motor that impels the anchor upwardly along the track or the anchor may be raised manually by an appropriate handle or strap to raise or lower the anchor. The patient's limb is lowered by reversing the movement of the anchor. Successive repetitions of this limb raising and lowering movement will provide the required therapy. By tilting the plane of the track the limb movement will have an angular component different than the straight upward raise when the plane of the track is perpendicular to the platform. Variations on these angular manipulations of the limb may be achieved by rotating the bed portion of the platform about its vertical axis.

### DESCRIPTION OF THE DRAWINGS

FIG. 1 is a perspective view of the therapeutic stretching table of the present invention showing a patient in supine position on the table with his right limb attached to the tension member that is attached to the movable anchor.

FIG. 2 is a perspective view of the “lazy Susan” type of interconnection between the floor standing frame and the bed portion of the platform.

FIG. 3 is a side view of the platform on which a patient is supported and the anchor carrying track that arches over the platform.

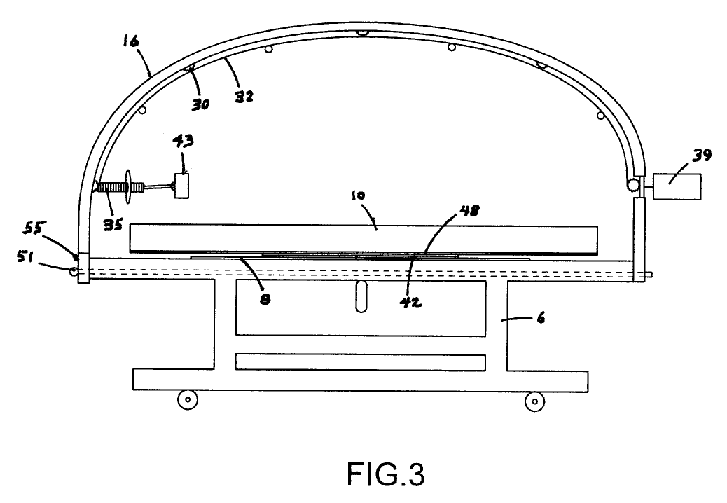

FIG. 4 is a top view of the table showing in dotted lines exemplary tilting positions of the track on either side of a position where the plane of the track is perpendicular to the platform and also showing the platform in an angular position with respect to the track.

FIG. 5 is an end view of the table showing the track in a tilted position away from the direct overhead perpendicular position.

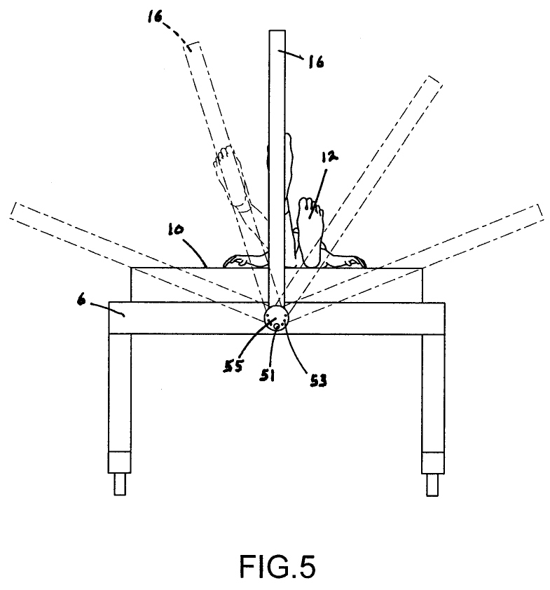

### DETAILED DESCRIPTION OF PREFERRED EMBODIMENT

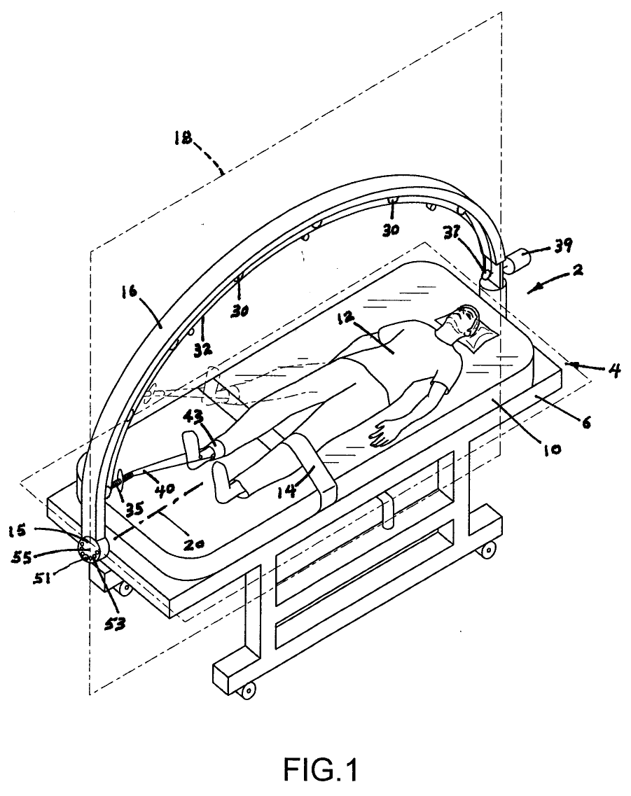

Referring first to FIG. 1, the therapeutic stretching table of the present invention is generally indicated by reference numeral 2. A platform 4 comprises a floor standing frame 6 having a flat top 8 on which is superimposed a bed 10 for supporting a patient 12. The platform, including the frame and bed portions, has a longitudinal extent that corresponds with the height or length of a supine patient. Attached to either the bed portion or the frame is a strap 14 or similar devise for securing the patient to the bed in a fixed position during the treatment provided by the apparatus of the present invention.

Pivotally attached to the longitudinal ends of the frame 6 are the terminal ends 15 of a track 16. The track is generally arch shaped and is contained within a single plane 18. The plane of the track intersects the center of the bed 10 along its longitudinal axis 20 when the bed is in a neutral or centered position, as shown in FIG. 1.

The track 6 is preferably comprised of a channel or similar structure that supports a sprocket and chain arrangement or a pulley and cable combination that can carry an anchor ring in movement along the channel. FIG. 1 depicts sprockets 30 that carry an endless loop chain 32. Attached to the chain is an anchor ring 35. The sprocket 37 at the head end of the track is connected in a traditional manner to a motor 39. Actuation of the motor will turn the sprocket in either of two directions to rotate the chain 32 and move the anchor ring 35 upwardly or downwardly along the channel that comprises the track 6. As an alternative to the motor drive, a suitable strap or handle can be connected to the anchor in order to allow manual movement of the anchor along the track.

The anchor ring 35 is connected to the proximal end of a tensioning mechanism 40 that has a distal end 43 adapted to be attached to the limb of a patient. Once attached and with the patient secured in place by the strap 14, the tensioning device 40 may be adjusted to produce as much tension on the limb as is desired or required for the subsequent manipulation. After the correct tension is applied the therapist operates the motor 39 to move the chain 32 and raise the anchor loop 35 which in turn raises the limb of the patient. The direction of the motor is reversed when the limb reaches the desired extension and the limb is lowered to whatever level is desired. The operation may be repeated as many times as desired.

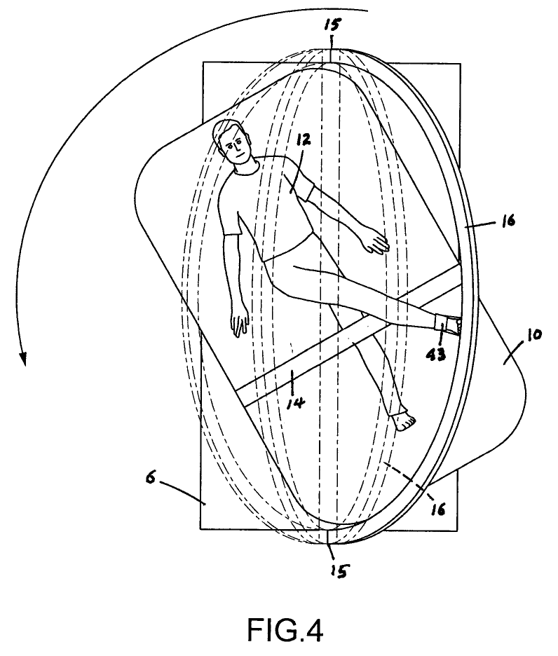

If the manipulation requires that the limb be lifted angularly instead of straight up, the track may be tilted by removing the locking pin 51 that is inserted into one of the apertures 53 in the hub 55 and a corresponding aperture in the frame top 8. Once the locking pin is removed the track may be tilted to an angular position either side of the normal position where the plane of the track is perpendicular to the bed 10. The angular position is secured by placing the locking pin 52 into an aperture in the hub that is aligned with the single aperture in the frame top 8. The angular position of the track will allow the limb to be manipulated at an angle instead of just straight up and down, as shown in FIG. 4.

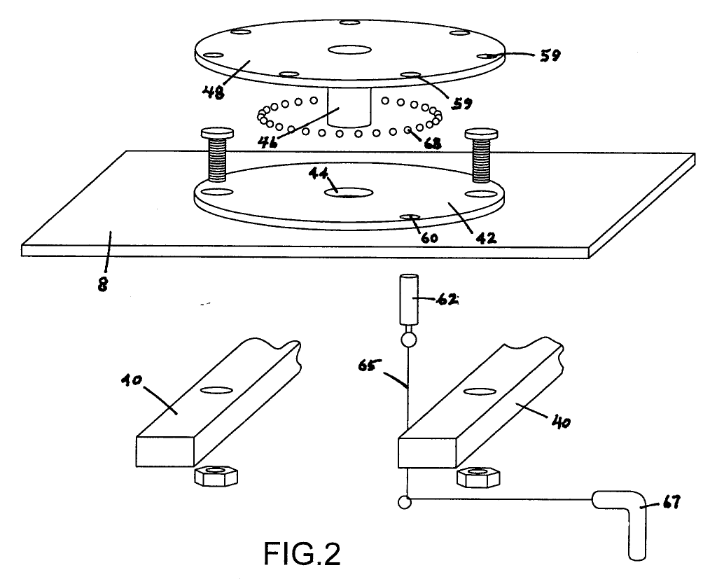

Further variations on adducting or abducting a limb in combination with raising and lowering the limb can be obtaining by rotating the bed 10 angularly with respect to the track 16, as shown in FIG. 4. Preferably, this rotation can be accomplished by mounting the bed 10 on a Lazy Susan type of mount, as illustrated in FIG. 2. The flat top 8 is bolted or otherwise secured to transverse members 40 of the frame 6. A circular steel base plate 42 is attached to the flat top and contains a centrally located journal 44 to receive the shaft 46 that depends from the rotatable plate 48 that is attached to the lower side of the bed 10. Indexing apertures 59 in the rotatable plate are positioned to align with a fixed aperture 60 in the steel base plate 42. A spring biased pin 62 inserted into one of the indexing apertures and the fixed aperture will establish the angular position of the bed with respect to the frame and the overhead track. In changing angular positions the rotatable plate 48 rotates freely on ball bearings 68 disposed between the base plate and the rotatable plate. The pin 62 may be operated by a cable 65 and a pull handle 67 appropriately attached to the frame.

What is claimed is:

1. Therapeutic apparatus comprising, platform means having a longitudinal aspect for supporting a human body,
an arch shaped track having first and second ends and lying in a plane, the track being superimposed over the platform whereby the plane of the track intersects the platform longitudinally,
means connecting the first and second ends of the track to the platform,
anchor means movably carried by the track, and
tension means having distal and proximal ends where the proximal end is attached to the anchor means.

2. The apparatus of claim 1 and further including motor means operably connected to the anchor means for moving the anchor means along the track.

3. The apparatus of claim 2 where the means connecting the first and second ends of the track to the platform are pivotal connections that allow the track to rotate in an arc, the diameter of which is a line interconnecting the first and second ends of the track.

4. The apparatus of claim 3 where the platform comprises,
a base member,
a bed member superimposed over the base member, and
means rotatably interconnecting the bed and the base members for angularly positioning the longitudinal aspect of the bed member with respect to the longitudinal aspect of the base member.
